(19) United States
(12) Patent Application Publication
(10) Pub. No.: US 2022/0323505 A1
(43) Pub. Date: Oct. 13, 2022

(54) METHODS FOR MAKING AUDITORY PROGENITOR CELLS AND USES THEREOF

(71) Applicant: The Regents of the University of California, Oakland, CA (US)

(72) Inventors: Alireza MOSHAVERINIA, Los Angeles, CA (US); Sevda POURAGHAEI SEVARI, Newbury Park, CA (US)

(73) Assignee: The Regents of the University of California, Oakland, CA (US)

(21) Appl. No.: 17/640,267

(22) PCT Filed: Sep. 11, 2020
(86) PCT No.: PCT/US20/50468

§ 371 Date: Mar. 3, 2022

Related U.S. Application Data
(60) Provisional application No. 62/899,570, filed on Sep. 12, 2019.

(51) Int. Cl. A61K 35/28; C12N 5/00; C12N 5/0793

(52) U.S. Cl. CPC A61K 35/28; C12N 5/0012; C12N 5/062; C12N 2506/1361; C12N 2501/115; C12N 2501/105; C12N 2501/11; C12N 2501/155; C12N 2533/20; C12N 2533/74

(57) ABSTRACT

Methods are described for preparing auditory progenitor cells from gingival mesenchymal cells, for uses such as restoring hearing in hearing impaired individuals. In one aspect, a method of treating hearing loss associated with loss of sensory neurons in a human subject is provided, the method comprising the steps of: a. obtaining a population of gingival mesenchymal stem cells (GMSCs); b. optionally expanding the population of GMSCs in vitro; c. encapsulating the population of GMSCs in an elastic three-dimensional scaffold; d. exposing the encapsulated population of GMSCs to a composition comprising one or more growth factors; e. allowing co a sufficient period for the population of GMSCs to differentiate towards auditory progenitor cells; f. optionally retrieving the auditory progenitor cells from the scaffold; and g. introducing the auditory progenitor cells into the inner ear of the subject.

### CROSS REFERENCE TO RELATED APPLICATION

This application claims benefit of U.S. Provisional Patent Application No. 62/899,570, filed Sep. 12, 2019, which is incorporated by reference herein in its entirety.

### GOVERNMENT SUPPORT

This invention was made with government support under DE023825, awarded by the National Institutes of Health. The Government has certain rights in the invention.

### BACKGROUND

Hearing loss is the most common sensory disability in humans, impairing the normal communication of more than 5% of population in industrialized societies. The core component of the cochlea, i.e., the organ of Corti, contains a highly ordered cellular mosaic of sensory hair cells (HCs) and non-sensory supporting cells (SCs). Once the sensory HCs are lost, the individual is shut off irreversibly from the hearing world. According to the World Health Organization (WHO), 466 million individuals are suffering from disabling hearing loss worldwide, and this number is estimated to exceed 900 million individuals by 2050. Hearing loss has a number of causes including genetic diseases, birth abnormalities, infections, certain medications, aging, and exposure to loud noises.

Drug therapies (e.g., corticosteroids) and implantable auditory devices are among the current treatment modalities used to combat sensorineural hearing loss, with limited success in treating acute cases; however, there is not any effective medical intervention available for treating chronic hearing loss. A growing body of biotechnological approaches including stem cell and gene therapy have emerged recently and have the potential to regenerate the damaged tissue. However, numerous limitations and obstacles have thus far prevented these developments from being translated into clinical application. These limitations include a lack of ready access to the necessary number of stem cells and concerns over the long-term viability of gene therapy. Therefore, an effective method for the treatment of hearing loss is yet to be discovered.

It is toward developing a treatment for hearing loss and the formation and utilization of auditory progenitor cells that the present invention is directed.

### SUMMARY OF THE INVENTION

In one aspect, a method of treating hearing loss associated with loss of sensory neurons in a human subject is provided, the method comprising the steps of:

a. obtaining a population of gingival mesenchymal stem cells (GMSCs);

b. optionally expanding the population of GMSCs in vitro;

c. encapsulating the population of GMSCs in an elastic three-dimensional scaffold;

d. exposing the encapsulated population of GMSCs to a composition comprising one or more growth factors;

e. allowing a sufficient period for the population of GMSCs to differentiate towards auditory progenitor cells;

f. optionally retrieving the auditory progenitor cells from the scaffold; and

g. introducing the auditory progenitor cells into the inner ear of the subject.

In one embodiment, the population of GMSCs are obtained from gingival tissue of a donor. In one embodiment, the donor is the human subject.

In one embodiment, the elasticity of the scaffold is a Young's modulus between about 1 kPa and about 30 kPa. In one embodiment, the elasticity of the scaffold is a Young's modulus is between about 1 kPa and about 12 kPa. In one embodiment, the elasticity of the scaffold is a Young's modulus is between about 1 kPa and about 6 kPa. In one embodiment, the elasticity of the scaffold is equal to or below an elastic modulus calculated from the slope of the initial portion of the loading curve of RGD-ALG/10-MG depicted in FIG. 3C. In one embodiment, the elasticity of the scaffold is equal to or below an elastic modulus calculated from the slope of the initial portion of the loading curve of RGD-ALG/20-MG depicted in FIG. 3C.

In one embodiment, the one or more growth factors are selected from basic fibroblast growth factor (bFGF), insulin-like growth factor 1 (IGF-1), epidermal growth factor (EGF), BMP4, FGF-2, SB-431542, CHIR99021, LDN-193189 or any combination thereof.

In one embodiment, the one or more growth factors are a combination of basic fibroblast growth factor (bFGF), insulin-like growth factor 1 (IGF-1), and epidermal growth factor (EGF).

In one embodiment, the scaffold comprises a hydrogel. In one embodiment, the hydrogel is a nanocomposite hydrogel. In one embodiment, the hydrogel comprises RGD peptide. In one embodiment, the hydrogel comprises alginate crosslinked with a divalent cation. In one embodiment, the hydrogel comprises RGD-alginate. In one embodiment, the hydrogel comprises MATRIGEL. In one embodiment, the hydrogel comprises about at least 10% MATRIGEL. In one embodiment, the hydrogel comprises about 10% to about 20% MATRIGEL. In one embodiment, the hydrogel comprises RDG-alginate and at least 10% MATRIGEL. In one embodiment, the hydrogel comprises RDG-alginate and about 10% to about 20% MATRIGEL.

In one embodiment, the GMSCs lack expression of CD34 or CD45, express CD76, CD146, or OCT4, or any combination thereof.

In one embodiment, the auditory progenitor cells express GATA3, SOX2, PAX2, PAX8, or any combination thereof.

In one embodiment, the auditory progenitor cells are injected into the auditory nerve trunk in the internal auditory meatus of the human subject in need thereof, thereby treating the hearing loss in the subject.

In one aspect, an in vitro method of generating auditory progenitor cells is provided, the method comprising the steps of:
a. obtaining a population of gingival mesenchymal stem cells (GMSCs);
b. optionally expanding the population of GMSCs in vitro;
c. encapsulating the population of GMSCs in an elastic three-dimensional scaffold;
d. exposing the encapsulated population of GMSCs to a composition comprising one or more growth factors;
e. allowing a sufficient period for the population of GMSCs to differentiate towards auditory progenitor cells; and
f. retrieving the auditory progenitor cells from the scaffold.

In one embodiment, the population of GMSCs are obtained from gingival tissue of a mammalian donor. In one embodiment, the donor is human.

In one embodiment, the elasticity of the scaffold is a Young's modulus between about 1 kPa and about 30 kPa. In one embodiment, the elasticity of the scaffold is a Young's modulus is between about 1 kPa and about 12 kPa. In one embodiment, the elasticity of the scaffold is a Young's modulus is between about 1 kPa and about 6 kPa. In one embodiment, the elasticity of the scaffold is equal to or below an elastic modulus calculated from the slope of the initial portion of the loading curve of RGD-ALG/10-MG depicted in FIG. 3C. In one embodiment, the elasticity of the scaffold is equal to or below an elastic modulus calculated from the slope of the initial portion of the loading curve of RGD-ALG/20-MG depicted in FIG. 3C.

In one embodiment, the one or more growth factors are selected from basic fibroblast growth factor (bFGF), insulin-like growth factor 1 (IGF-1), epidermal growth factor (EGF), BMP4, FGF-2, SB-431542, CHIR99021, LDN-193189 or any combination thereof.

In one embodiment, the one or more growth factors are a combination of basic fibroblast growth factor (bFGF), insulin-like growth factor 1 (IGF-1), and epidermal growth factor (EGF).

In one embodiment, the scaffold comprises a hydrogel. In one embodiment, the hydrogel is a nanocomposite hydrogel. In one embodiment, the hydrogel comprises RGD peptide. In one embodiment, the hydrogel comprises alginate crosslinked with a divalent cation. In one embodiment, the hydrogel comprises RGD-alginate. In one embodiment, the hydrogel comprises MATRIGEL. In one embodiment, the hydrogel comprises about at least 10% MATRIGEL. In one embodiment, the hydrogel comprises about 10% to about 20% MATRIGEL. In one embodiment, the hydrogel comprises RDG-alginate and at least 10% MATRIGEL. In one embodiment, the hydrogel comprises RDG-alginate and about 10% to about 20% MATRIGEL.

In one embodiment, the GMSCs lack expression of CD34 or CD45, express CD76, CD146, or OCT4, or any combination thereof.

In one embodiment, the auditory progenitor cells express GATA3, SOX2, PAX2, PAX8, or any combination thereof.

In one embodiment, the auditory progenitor cells are injected into the auditory nerve trunk in the internal auditory meatus of a human subject in need thereof, thereby treating hearing loss in the subject. In one embodiment, the human subject is the donor.

In another aspect, a method is provided for treating hearing loss in a subject comprising the steps of: (a) preparing auditory progenitor cells in accordance with the foregoing methods; and (b) introducing the auditory progenitor cells into the inner ear of the subject. In one embodiment, the population of gingival mesenchymal stem cells used in the method of preparing auditory progenitor cells are from the subject.

These and other aspects of the invention will be apparent from the ensuing brief description of the drawings and detailed description of the invention.

### BRIEF DESCRIPTIONS OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. Depicts a schematic representation of differentiating the encapsulated GMSCs toward auditory progenitor cells.

FIG. 2A, FIG. 2B, FIG. 2C and FIG. 2D show the characterization of the isolated human GMSCs. FIG. 2A depicts immunofluorescent staining showing positive staining for the sternness markers, CD146 and OCT4. FIG. 2B shows expression of MSC surface markers on GMSCs evaluated using flow cytometric analysis. FIG. 2C shows fluorescence images of Live/dead staining of the encapsulated GMSCs in fabricated hydrogels after seven days of culturing in regular media, scale bars 250 μm. FIG. 2D shows quantitative live/dead results of encapsulated MSCs shows not significant difference between the viability of MSCs encapsulated in the different hydrogels (p>0.05).

FIG. 3A, FIG. 3B, FIG. 3C and FIG. 3D show the characterization of the fabricated hydrogels. FIG. 3A shows a SEM image of freeze-dried hydrogels representing a homogenous macroporous microstructure, scale bar 100 μm. Inserts are magnified images for better demonstration, scale bars 20 μm. FIG. 3B depicts the value of the measured effective Young's modulus. FIG. 3C is a force vs. displacement graph obtained from indenting samples Alg-10MG and Alg-20MG after cross-linking. FIG. 3D are elasticity maps, Alg-20MG (left) and Alg-10MG (right).

FIG. 4A, FIG. 4B, FIG. 4C and FIG. 4D show the characteristics of GMSCs. FIG. 4A depicts phase contrast images showing the GMSCs encapsulated in the alginate hydrogel containing two different concentrations of Matrigel (20MG and 10MG), 20% and 10%, respectively, or without Matrigel after 1, 2, and 4 weeks of incubation at the inductive culture media. The images demonstrate successful formation of numerous neurospheres after 4 weeks of encapsulation in the alginate hydrogel containing higher concentration of Matrigel (20%). FIG. 4B shows RT-PCR results studying expression of the preplacodal ectoderm genes, GATA3 and SOX2, and the early otic markers Pax2 and Pax8. FIG. 4C shows Western blot analysis presented changes in the expression levels of auditory progenitor regulators in GMSCs with higher level of GATA3, SOX2, PAX2 and PAX8 expression in the GMSCs encapsulated in the alginate hydrogel containing higher concentration of the Matrigel (20MG) in comparison to the hydrogel containing 10% Matrigel (10MG) or the alginate without Matrigel (Ctrl). FIG. 4D depicts semi-quantitative analysis of the relative band intensity of the western blots. (NS: not significant. *p<0.05, **p<0.01, and ***p<0.001).

FIG. 5A and FIG. 5B depict characteristics of GMSCs. FIG. 5A depicts immunofluorescence staining of the GMSCs encapsulated in the nanocomposite hydrogel containing two different amounts of Matrigel (10%, 20%) or without Matrigel (control). GATA3 and SOX2 were used as preplacodal ectoderm markers. Specimens were counterstained with DAPI. Scale bar 25 μm. FIG. 5B is a semi-quantitative analysis of double-positive staining expressing GATA3-Sox2. (NS: not significant. *p<0.05).

FIG. 6A and FIG. 6B depict otic differentiation marker expression by subcutaneous implantation of the encapsulated GMSCs. FIG. 6A depicts subcutaneous implantation of the encapsulated GMSCs in Alg-20MG hydrogel (Alg-20MG/GMSCs), the cell free hydrogels (Control) were implanted as the negative control. The dashed lines are indicating the hydrogel beads. FIG. 6A shows immunofluorescence staining for otic differentiation markers SOX2 and PAX8 (red) and GATA3 and PAX2 (green). The cellular nucleus was counterstained with DAPI (blue); and FIG. 6B depicts semi-quantitative analysis of the percentage of positive cells for Sox2, GATA3, PAX8, and PAX2 antibodies according to the immunostaining results of FIG. 6A.

FIG. 7A, FIG. 7B and FIG. 7C depict otic differentiation marker expression in encapsulated GMSCs. FIG. 7A shows immunofluorescence staining of the GMSCs encapsulated in the nanohybrid hydrogel containing two different amounts of Matrigel (10%, 20%) or without Matrigel (control). Pax2 and Pax8 were used as early otic markers. The specimens were counterstained with DAPI. Scale bar 25 μm. FIG. 7B is a schematic illustration showing encapsulation of the GMSCs in alginate-Matrigel nanohybrid hydrogel in presence of EGF, IGF-I, and bFGF growth factors. FIG. 7C shows semi-quantitative analysis of double-positive staining expressing PAX8-PAX2. (*p<0.05, **p<0.01, and ***p<0.001).

FIG. 8A, FIG. 8B and FIG. 8C depict otic differentiation marker expression of encapsulated GMSCs. FIG. 8A shows immunofluorescence staining of the GMSCs encapsulated in the nanohybrid hydrogel containing two different amounts of Matrigel (10%, 20%) or without Matrigel (control). Pax8 and Myo7a were used as otic markers. The specimens were counterstained with DAPI. Scale bar 25 μm. FIG. 8B shows semi-quantitative analysis of double-positive staining expressing PAX8-PAX2. (*p<0.05, **p<0.01, and ***p<0.001). FIG. 8C is a schematic illustration of regenerating auditory progenitor cells by GMSCs encapsulated in the engineered nanohybrid hydrogels in presence of EGF, IGF-I, and bFGF growth factors.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Success in tissue engineering relies on the appropriate selection of the cell source, scaffolding material, and growth factors. A source of neural and sensory cells would therefore be a valuable tool for regeneration of auditory system Gingival mesenchymal stem cells (GMSCs) are highly promising in this regard since they can be obtained easily from oral tissue or, for allogeneic use, from dental clinics as discarded biological samples.

Using a scaffold of a certain elasticity, in one non-limiting embodiment RGD-modified alginate/Matrigel nanocomposite hydrogel, and in the presence of growth factors, the inventors herein demonstrated the induction of auditory progenitors from human GMSCs.

GMSCs were used to identify the proper microenvironment for developing otic progenitors. In one embodiment, the potential of GMSCs to differentiate toward auditory progenitors was demonstrated when GMSCs were encapsulated in a soft 3D nanocomposite hydrogel when given the proper soluble signals. In vivo studies further demonstrated the feasibility of utilizing GMSCs as a cell source for generating auditory progenitor cells. Moreover, the studies described herein shows that signals from the microenvironment can regulate stem cell fate and gene expression. The results confirmed that altering the biophysical properties of the microenvironment could affect the ability of the ectoderm to respond properly to soluble signals.

Sensorineural hearing loss in mammals occurs due to irreversible damage to the sensory epithelia of the inner ear and has very limited treatment options. The ability to regenerate the auditory progenitor cells is a promising approach for the treatment of sensorineural hearing loss; therefore, finding an appropriate and easily accessible stem cell source for restoring the sense of hearing would be of great interest. Gingival mesenchymal stem cells (GMSCs) are characterized by high self-renewal and multipotent differentiation capacity and are easily accessible from the oral cavity or discarded tissue samples at dental clinics.

In one embodiment, the biophysical properties of the cellular microenvironment play a critical role in stem cell function and lineage commitment. GMSCs in combination with an appropriate scaffold material can therefore present advantageous therapeutic options for a number of conditions. GMSCs have the potential to differentiate into auditory progenitor cells in the presence of a three-dimensional scaffold and certain growth factors. In one embodiment, encapsulated GMSCs within a nanocomposite hydrogel composed of RGD-coupled alginate and Matrigel and incubated them in specific growth medium containing fibroblast growth factor-basic (bFGF), insulin-like growth factor (IGF) and epidermal growth factor (EGF) resulted in the formation of auditory progenitor cells. Moreover, the in vitro studies confirmed the potential of GMSCs to differentiate toward auditory progenitors in this microenvironment. In one embodiment, the elasticity of the scaffold, in one non-limiting example a composition comprising Matrigel and alginate hydrogel in the three-dimensional scaffold, altered the previously demonstrated mechanical properties of the alginate and enabled the differentiation of GMSCs toward and into auditory progenitor cells. This observation was confirmed using in vivo studies showing the ability of the GMSCs to develop into auditory progenitor cells in this suitable microenvironment.

Each of the steps of the methods herein is elaborated upon below.

Source of GMSCs. Human GMSCs are present in gingival tissues and can be obtained from tissue obtained during oral surgery according to published methods. As noted herein, a subject in need of therapy for hearing loss can donate gingival tissue for preparing auditory progenitor cells for implantation of autologous cells. In other embodiments, donors can be from one or more different subjects.

Characterization of GMSCs. The characteristics of isolated GMSCs can be determined by, for example, using flow cytometric analysis according to the methods in the literature. In one embodiment, the lack of expression of hematopoietic stem cell markers (such as but not limited to CD34 and CD45) and positive expression of MSC surface markers such as but not limited to CD76 and CD146 characterize GMSCs. In one embodiment, immunofluorescence (IF) staining of a 2D monolayer culture of the isolated GMSCs can be used to confirm expression of sternness markers such as but not limited to CD146 and OCT4.

Expanding GMSCs in vitro. Prior to the subsequent step, the GMSCs may be expanded in vitro. In one embodiment, the cells are expanded up to passage two, three, four, five or more. Methods for expansion are well known in the art. In one example, GMSCs are cultured in α-MEM (Life Technologies), 10% FBS, 1% Glutamax (Invitrogen) and 100 units/ml penicillin/streptomycin (100 μg/ml, Sigma) at 37° C. in a 5% CO2 incubator and expanded up to passage four. In one embodiment, cells are trypsinized and collected by centrifugation.

Three-dimensional scaffolds. As described herein, the GMSCs are encapsulated in a scaffold in which differentiation toward auditory progenitor cells occurs. Any of a selection of scaffold matrices may be used, such as but not limited to alginate, MATRIGEL, gelatin, and collagen. Other scaffolds that can be used include Geltrex® BME PathClear, Geltrex® Matrix, vitronectin, BioGelx RGD, Cellendes, HyStem, MatriXpex 3D and Xylyx, by way of non-limiting examples. As described elsewhere herein, the elasticity of the scaffold aids in the differentiation of the GMSCs towards auditory progenitor cells.

Alginate is a highly biocompatible natural polysaccharide derived from seaweed. Alginates are known to be block copolymers consisting of blocks of (1,4)-linked β-D-mannuronate (M) and α-L-guluronate (G) residues. Alginate hydrogels can be obtained by crosslinking the G-blocks along with the polymer with divalent cations like Ca²⁺ (A. Moshaverinia, C. Chen, K. Akiyama, S. Ansari, X. Xu, W. W. Chee, S. R. Schricker, S. Shi, Alginate hydrogel as a promising scaffold for dental-derived stem cells: an in vitro study, J. Mater. Sci. Mater. Med. 23 (2012) 3041-3051. doi:10.1007/s10856-012-4759-3; K. Y. Lee, D. J. Mooney, Alginate: properties and biomedical applications., Prog. Polym. Sci. 37 (2012) 106-126. doi:10.1016/j.progpolymsci.2011.06.003). The resulting hydrogels possess a 3D structure that is stable over time, but they are unable to interact with mammalian cells due to a lack of cell adhesion ligands. Modifying the alginate with RGD sequences can rectify this and provide a suitable 3D structure for the prolonged culture of cells (J. A. Rowley, G. Madlambayan, D. J. Mooney, Alginate hydrogels as synthetic extracellular matrix materials, Biomaterials. 20 (1999) 45-53. http://www.ncbi.nlm.nih.gov/pubmed/9916770).

Matrigel is a mixture of different proteins obtained from the Engelbreth—Holm-Swarm tumor cell line that can mimic the basement membrane found in different tissues (H. K. Kleinman, G. R. Martin, Matrigel: Basement membrane matrix with biological activity Semin. Cancer Biol. 15 (2005) 378-386). It has been shown that the nano-scale topographic features of commercially available Matrigel are very similar to the native basement membrane, but its structural weakness has limited its application to monolayer or thin layer gel cultures. The elastic modulus of Matrigel can range from 120-450 Pa, depending on the temperature; Matrigel polymerizes and stiffens above 15° C.

As described herein, the elasticity of the scaffold aids in the differentiation of the GMSCs toward auditory progenitor cells. A scaffold of the appropriate elasticity can be made in a variety of ways, using the aforementioned matrices, following guidance such as that provided by O. Chaudhuri, S. T. Koshy, C. Branco da Cunha, J.-W. Shin, C. S. Verbeke, K. H. Allison, D. J. Mooney, Extracellular matrix stiffness and composition jointly regulate the induction of malignant phenotypes in mammary epithelium, Nat. Mater. 13 (2014) 970-978. In the examples below, we identified a range of elasticities of the scaffold that enables differentiation to occur. While not wishing to be bound by theory, the scaffold of the invention was made to mimic the elasticity of the region of the cochlea where auditory cells develop and function. In the studies herein, the scaffolds were prepared over the range of about 1 kPa to about 30 kPa. In certain embodiments, the elasticity found improve differentiation was below about 12 kPa, and in other embodiments, below about 6 kPa. In some embodiments, an elasticity of about 1 kPa to about 2 kPa was found to improve differentiation. This, in one non-limiting embodiment, the range of elasticity of from about 1 kPa to about 6 kPa was employed.

In one non-limiting example, to prepare a 3D nanocomposite hydrogel based on alginate and Matrigel, reconstituted GRGDSP-coupled high G high MW Alginate, G/M Ratio: >1.5, (FMC biopolymer, NovaMatrix®, Norway) is reconstituted in Dulbecco's PBS at a concentration of 1.25% wt/v with shaking overnight at room temperature. Growth Factor Reduced Matrigel (Corning®, Life Sciences) is thawed overnight at 4° C. Two different concentrations of Matrigel, 10% v/v and 20% v/v, may be used to form nanocomposite hydrogels (referred to here as Alg-10MG and Alg-20MG, respectively). The RGD-Alginate without Matrigel is used as the control. To form the hydrogel, the reconstituted RGD-Alginate with the concentration of 1.25% wt/v is delivered into a 3 ml syringe and put on ice to cool down before mixing with Matrigel. The appropriate amount of Matrigel was delivered into another syringe to reach to a final concentration of 10% v/v or 20% v/v in Alg-10MG and Alg-20MG compositions, respectively. Next, the two syringes were locked with a female Luer adapter (Cole-Parmer), mixed very quickly, and again put on ice to avoid gelation of the Matrigel. The well-mixed presolutions were cross-linked using 10 mM Calcium Sulfate. Cross-linking with lower concentrations of Calcium Sulfate is a slow reaction that gives enough time for properly mixing the solution before complete cross-linking.

To evaluate the microstructure of the hydrogel nanocomposites with scanning electron microscopy (SEM; Zeiss Supra 40VP), the samples are freeze-dried (FreeZone® Benchtop Freeze Dryers, Labconco®) and coated with Iridium (South Bay Technology Ion Beam Sputtering/Etching System) before imaging.

Elasticity measurement of scaffolds. Nanoindentation tests to determine elasticity can be carried out using the displacement-controlled Piuma Nanoindenter (Optics11, Amsterdam, The Netherlands). A force sensing probe containing a 50 μm radius spherical borosilicate glass tip and a cantilever stiffness of 0.5 N/m were used to test the samples. Measurements can be performed by gluing samples onto the bottom of a Petri dish and then submerging them in PBS at room temperature with the nanoindenter tip remaining well below the surface of water at all times in order to avoid any error introduced by strong adhesive forces at the air-water interface. The effective elastic modulus (E_eff) can be calculated using the slope of the initial portion of the loading curve using the Hertz theory shown in the following equations:

1/E_eff≈(1−v2)/E
where E is the sample elastic modulus defined by: E=(3(1−v\hat{ }2)P)/(4R\hat{ }1/2h\hat{ }3/2).

Encapsulating GMSCs in the scaffold. Encapsulation of GMSCs inside the hydrogel scaffold may be performed according to previously published methods, such as but not limited to A. Moshaverinia, C. Chen, K. Akiyama, X. Xu, W. W. L. Chee, S. R. Schricker, S. Shi, Encapsulated dental-derived mesenchymal stem cells in an injectable and biodegradable scaffold for applications in bone tissue engineering, J. Biomed. Mater. Res. Part A. (2013) n/a-n/a. doi:10.1002/jbm.a.34546; and S. Ansari, C. Chen, X. Xu, N. Annabi, H. H. Zadeh, B. M. Wu, A. Khademhosseini, S. Shi, A. Moshaverinia, Muscle Tissue Engineering Using Gingival Mesenchymal Stem Cells Encapsulated in Alginate Hydrogels Containing Multiple Growth Factors, Ann. Biomed. Eng. 44 (2016) 1908-1920. doi:10.1007/s10439-016-1594-6. In one example, GMSCs are cultured on a 10 cm cell-culture-treated plate in α-MEM (Life Technologies), 10% FBS, 1% Glutamax (Invitrogen) and 100 units/ml penicillin/streptomycin (100 μg/ml, Sigma) at 37° C. in a 5% CO2 incubator and expanded up to passage four. At this point, cells are trypsinized and centrifuged. The pelleted GMSCs are mixed with the RGD-Alginate/Matrigel obtained from the previous step at a concentration of 10⁶ cells/ml in two 3-ml syringes locked with a female Luer adapter. 10 mM Calcium Sulfate was used to cross-link the hydrogel encapsulating the GMSCs.

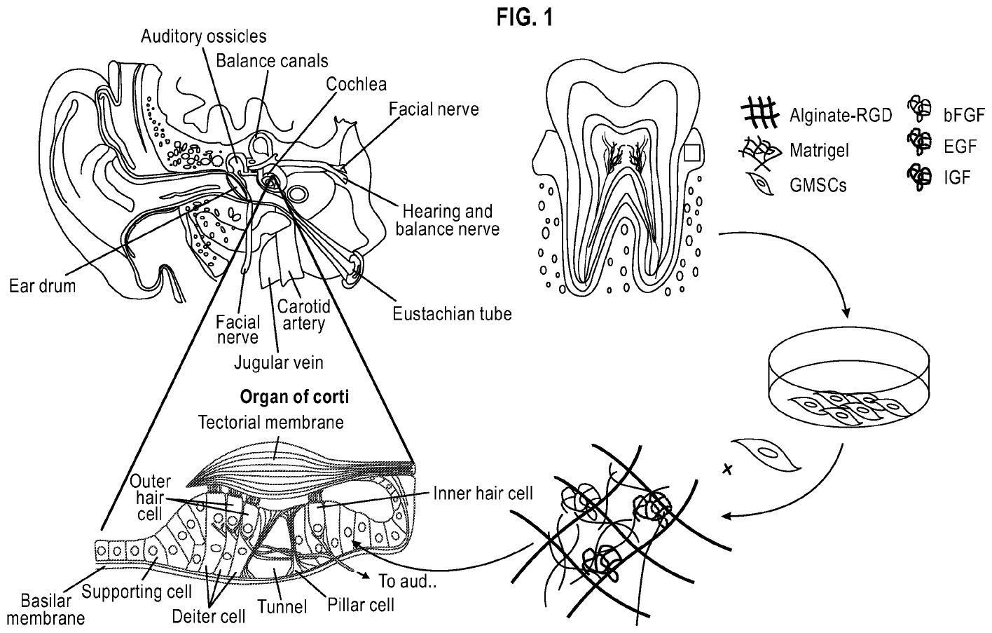

Exposing GMSCs to growth factors. In combination with the appropriate elasticity of the scaffold described above, one or more growth factors may be included in the scaffold to aid in the differentiation of the GMSCs towards auditory progenitor cells. In one non-limiting example, growth factors such as basic fibroblast growth factor (bFGF), insulin-like growth factor 1 (IGF-1), epidermal growth factor (EGF), BMP4, FGF-2, SB-431542, CHIR99021, LDN-193189 or any combination thereof may be used. In one embodiment, a combination of basic fibroblast growth factor (bFGF), insulin-like growth factor 1 (IGF-1), epidermal growth factor (EGF) may be used. In one example, for auditory progenitor induction, serum-free DMEM/F12 1:1 medium supplemented with N2/B27 (Invitrogen) can be modified with a combination of EGF (20 ng/ml) and IGF-1 (50 ng/ml; Gold Biotechnology). The cell-laden hydrogels as described above can be cast in 12-well plates and incubated in the prepared induction medium for two weeks followed by the addition of bFGF (10 ng/ml; Gold Biotechnology) plus the other growth factors (EGF and IGF-1) and incubated for an additional two weeks. The medium was changed every three days. FIG. 1 shows a schematic representation of the encapsulating GMSCs inside alginate/Matrigel hydrogels followed by auditory progenitor induction.

Thus, in one embodiment, the GMSCs are encapsulated in medium containing EGF and IGF-1, allowed to incubate for two weeks in medium containing EGF and IGF-1, then the encapsulated cells are incubated in a combination of EGF, IGF-1 and bFGF for an additional two weeks.

Differentiation of GMSCs to auditory progenitor cells. As noted herein, during the incubation of the cells in the scaffold, the GMSCs different toward auditory progenitor cells. The elasticity of the scaffold enables the differentiation to occur. While the growth factors used and the protocol used for induction is described herein, it is a non-limiting example of conditions to induce differentiation.

Characterization of auditory progenitor cells. The prosensory regions of the auditory system can be distinguished by the expression of specific sensory genes, such as described in B. Fritzsch, K. W. Beisel, L. A. Hansen, The molecular basis of neurosensory cell formation in ear development: a blueprint for hair cell and sensory neuron regeneration, Bioessays. 28 (2006) 1181-93. The family of Pax genes can be used to identify the otic prosensory region during vertebrate cranial placode development. Pax8 is one of the earliest and well-known markers of otic development in the vertebrate. Pax2 is known as another early marker of otic placode development which is actively expressed in different areas of the ear during subsequent development. During the development of the inner ear, the transcription factor GATA3 is expressed in the whole otic placode consisting of several different tissues, including neural and epithelial cells and periotic mesenchymal cells. Throughout the development of the inner ear, GATA3 and Pax2 are both expressed in the cochlea. Sox2, a high mobility group (HMG) box domain transcription factor known for maintaining pluripotency in progenitor and stem cells, also plays a vital role in neurogenesis and development of sensory progenitors. Expression of Sox2 is a prerequisite for the specification of sensory fate in the auditory system.

As noted herein, differentiation of cells encapsulated in nanocomposite hydrogels with different elasticity and composition show that a decrease in elastic modulus of the microenvironment can trigger differentiation of preplacodal ectoderm towards otic placode lineages. Overall, the best results were achieved through combining a relatively high concentration of Matrigel (Alg-20MG) with the necessary soluble factors, as confirmed by the gene and protein expression and IF staining results.

Retrieving auditory progenitor cells from the scaffold. For subsequent use of the auditory progenitor cells for implantation or evaluation, removal from the scaffold may be required. In one embodiment, the cell-laden hydrogels may be dissolved using a reagent that solubilizes alginate matrix, such as AlgiMatrix™ Dissolving Buffer (Life Technologies Corporation). Cells may be washed and stored. A composition or formulation comprising the auditory progenitor cells may be used for implantation, infusion, injection, or surgical placement for the treatment of hearing loss, or other purposes. In certain embodiments, the scaffold comprising the auditory progenitor cells may be used for such purposes without isolation or retrieval from the scaffold.

Uses of auditory progenitor cells. Treatment of hearing loss in human subjects or other mammalian species is one use of the auditory progenitor cells of the invention. Implantation of auditory progenitor cells derived from the subjects own GMSCs provides an autologous means of restoring hearing loss. In one embodiment, the auditory progenitor cells are injected into the auditory nerve trunk in the internal auditory meatus of a human subject in need thereof, thereby treating hearing loss in the subject.

The following examples are presented in order to more fully illustrate the preferred embodiments of the invention. It should in no way be construed, however, as limiting the broad scope of the invention.

### EXAMPLES

### Experimental Methods

Isolation and characterization of the GMSCs. Human GMSCs were extracted from gingival tissues of healthy patients according to published protocol (A. Moshaverinia, X. Xu, C. Chen, S. Ansari, H. H. Zadeh, M. L. Snead, S. Shi, Application of stem cells derived from the periodontal ligament or gingival tissue sources for tendon tissue regeneration, Biomaterials. 35 (2014) 2642-2650. doi:10.1016/j.biomaterials.2013.12.053; S. Ansari, C. Chen, X. Xu, N. Annabi, H. H. Zadeh, B. M. Wu, A. Khademhosseini, S. Shi, A. Moshaverinia, Muscle Tissue Engineering Using Gingival Mesenchymal Stem Cells Encapsulated in Alginate Hydrogels Containing Multiple Growth Factors, Ann. Biomed. Eng. 44 (2016) 1908-1920. doi:10.1007/s10439-016-1594-6). Isolated cells were expanded to passage four before use for studies in vitro. The sternness of the isolated GMSCs was confirmed using flow cytometric analysis according to the methods in the literature. The lack of expression of hematopoietic stem cell markers (CD34 and CD45) and positive expression of MSC surface markers CD76 and CD146 were confirmed. In addition, Immunofluorescence (IF) staining of a 2D monolayer culture of the isolated GMSCs confirmed expression of sternness markers CD146 (Abcam) and OCT4 (Proteintech).

Formation of RGD-modified Alginate-Matrigel nanocomposite hydrogels. To prepare a 3D nanocomposite hydrogel based on alginate and Matrigel, we first reconstituted GRGDSP-coupled high G high MW Alginate, G/M Ratio: >1.5, (FMC biopolymer, NovaMatrix®, Norway) in D-PBS at a concentration of 1.25% wt/v with shaking overnight at room temperature. Growth Factor Reduced Matrigel (Corning®, Life Sciences) was thawed overnight at 4° C. In order to better study the effect of Matrigel and also matrix elasticity on the differentiation of GMSCs toward auditory progenitor cells, two different concentrations of Matrigel, 10% v/v and 20% v/v, were used to form nanocomposite hydrogels (referred to here as Alg-10MG and Alg-20MG, respectively). The RGD-Alginate without Matrigel was used as the control. To form the hydrogel, the reconstituted RGD-Alginate with the concentration of 1.25% wt/v was delivered into a 3 ml syringe and put on ice to cool down before mixing with Matrigel. The appropriate amount of Matrigel was delivered into another syringe to reach to a final concentration of 10% v/v or 20% v/v in Alg-10MG and Alg-20MG compositions, respectively. Next, the two syringes were locked with a female Luer adapter (Cole-Parmer), mixed very quickly, and again put on ice to avoid gelation of the Matrigel. The well-mixed presolutions were cross-linked using 10 mM Calcium Sulfate. Cross-linking with lower concentrations of Calcium Sulfate is a slow reaction that gives enough time for properly mixing the solution before complete cross-linking.

To evaluate the microstructure of the hydrogel nanocomposites with scanning electron microscopy (SEM; Zeiss Supra 40VP), the samples were freeze-dried (FreeZone® Benchtop Freeze Dryers, Labconco®) and coated with Iridium (South Bay Technology Ion Beam Sputtering/Etching System) before imaging.

Micro-mechanical measurements of hydrogels. Alg-10MG and Alg-20MG were prepared as described above. Nanoindentation tests were carried out using the displacement-controlled Piuma Nanoindenter (Optics11, Amsterdam, Netherlands). A force sensing probe containing a 50 μm radius spherical borosilicate glass tip and a cantilever stiffness of 0.5 N/m were used to test the samples. All measurements were performed by gluing samples to the bottom of a Petri dish and then submerging them in PBS at room temperature with the nanoindenter tip remaining well below the surface at all times in order to avoid any error introduced by strong adhesive forces at the air-PBS interface. The effective elastic modulus (E_eff) was calculated using the slope of the loading curve.

GMSC encapsulation and differentiation into auditory progenitor cells inside the RGD-Alginate/Matrigel nanocomposite hydrogel. Encapsulation of GMSCs inside the hydrogels was performed according to previously published articles (A. Moshaverinia, C. Chen, K. Akiyama, X. Xu, W. W. L. Chee, S. R. Schricker, S. Shi, Encapsulated dental-derived mesenchymal stem cells in an injectable and biodegradable scaffold for applications in bone tissue engineering, J. Biomed. Mater. Res. Part A. (2013) n/a-n/a. doi:10.1002/jbm.a.34546; S. Ansari, C. Chen, X. Xu, N. Annabi, H. H. Zadeh, B. M. Wu, A. Khademhosseini, S. Shi, A. Moshaverinia, Muscle Tissue Engineering Using Gingival Mesenchymal Stem Cells Encapsulated in Alginate Hydrogels Containing Multiple Growth Factors, Ann. Biomed. Eng. 44 (2016) 1908-1920. doi:10.1007/s10439-016-1594-6). Briefly, GMSCs were cultured on a 10 cm cell-culture-treated plate in α-MEM (Life Technologies), 10% FBS, 1% Glutamax (Invitrogen) and 100 units/ml penicillin/streptomycin (100 μg/ml, Sigma) at 37° C. in a 5% CO2 incubator and expanded up to passage four. At this point, cells were trypsinized and centrifuged. The pelleted GMSCs were mixed with the RGD-Alginate/Matrigel obtained from the previous step at a concentration of 10⁶ cells/ml in two 3-ml syringes locked with a female Luer adapter (Cole-Parmer). 10 mM Calcium Sulfate was used to cross-link the hydrogel encapsulating the GMSCs.

For auditory progenitor induction, serum-free DMEM/F12 1:1 medium supplemented with N2/B27 (Invitrogen) was modified with a combination of EGF (20 ng/ml) and IGF-1 (50 ng/ml; Gold Biotechnology). The cell-laden hydrogels were cast in 12-well plates and incubated in the prepared induction medium for two weeks followed by the addition of bFGF (10 ng/ml; Gold Biotechnology) plus the other growth factors (EGF and IGF-1) and incubated for an additional two weeks. The medium was changed every three days. FIG. 1 shows a schematic representation of the encapsulating GMSCs inside alginate/Matrigel hydrogels followed by auditory progenitor induction.

Viability of the GMSCs encapsulated in the nanocomposite hydrogels. The viability of the GMSCs encapsulated in the hydrogels was analyzed using LIVE/DEAD™ Viability/Cytotoxicity Kit for mammalian cells (L3224, Invitrogen). The percentage of the live cells was measured using ImageJ software (NIH, Bethesda, Md.).

Quantitative Real-Time PCR. After four weeks of incubation, the cell-laden hydrogels were dissolved using AlgiMatrix™ Dissolving Buffer (Life Technologies Corporation), and the encapsulated GMSCs were retrieved. Total RNA of the cells was extracted by the Trizol reagent (Invitrogen) according to the manufacturer's instructions. Subsequently, 1 μg of total RNA was reverse transcribed using qScript cDNA SuperMix (Quantabio), and the generated cDNA was subjected to RT-PCR using the primer pairs listed below.

| Gene | Sense primer | Antisense primer |
|---|---|---|
| SOX2 | 5′-ATAATAACAATCATCATCGGCGG-3′ | 5′-AAAAAGAGAGAGGCAAACTG-3′ |
| | (SEQ ID NO: 1) | (SEQ ID NO: 2) |
| GATA-3 | 5′-GCTGTCTGCAGCCAGGAGAGC-3′ | 5′-ATGCATCAAACAACTGTGGCCA-3′ |
| | (SEQ ID NO: 3) | (SEQ ID NO: 4) |
| Pax2 | 5AACGACAGAACCCGACTATG-3 | 5ATCCCACTGGGTCATTGGAG-3′ |
| | (SEQ ID NO: 5) | (SEQ ID NO: 6) |
| Pax8 | 5′-CTGAGGGCGICTGTGACAATG-3′ | 5TGAATGGTTGCTGCACTTTGG-3′ |
| | (SEQ ID NO: 7) | (SEQ ID NO: 8) |
| Glyceraldehyde | 5′-AGCCGCATCTTCTTTTGCGTC-3′ | 5′-TCATATTTGGCAGGTTTTTCT-3′ |
| 3-phosphate | (SEQ ID NO: 9) | (SEQ ID NO: 10) |
| dehydrogenase | | |
| (GADPH) | | |

We studied the expression of a set of neural and pre-placodal ectoderm (PPE) markers (GATA3, SOX2) as well as early otic markers (PAX2, PAX8) during differentiation of the encapsulated cells. GAPDH was used as the reference housekeeping gene.

Western blot analysis. After 4 weeks of incubation in the auditory inductive culture media, Western blot analysis was carried out to evaluate the expression of auditory markers by the GMSCs, as previously reported [A. Moshaverinia, S. Ansari, C. Chen, X. Xu, K. Akiyama, M. L. Snead, H. H. Zadeh, S. Shi, Co-encapsulation of anti-BMP2 monoclonal antibody and mesenchymal stem cells in alginate microspheres for bone tissue engineering, Biomaterials. 34 (2013) 6572-6579. doi: 10.1016/j.biomaterials 0.2013.05.048]. Briefly, the encapsulated cells were retrieved after dissolving the hydrogel with AlgiMatrix™ Dissolving Buffer (Life Technologies Corporation) and were lysed by RIPA lysing system (Santa Cruz Biotechnology). The protein concentration was measured by Pierce™ Coomassie (Bradford) Protein Assay Kit (Thermo Scientific™) according to the manufacturer's instructions. Next, 30 μs of each protein sample was subjected to SDS-PAGE followed by transferring onto nitrocellulose membranes (Bio-Rad). Then, the membranes were incubated with antibodies against GATA3 (1:500), SOX2 (1:500), PAX8 (1:2000) (Proteintech Group, Inc.), and PAX2 (1:500, Invitrogen). β-Tubulin (1:2000, Abcam) served as the housekeeping reference. Immune-protein complexes were probed with HRP Conjugated Anti-Rabbit IgG (H+L) antibody (Promega Corporation). Intensity of the acquired bands was quantified with the NIH ImageJ software (NIH, Bethesda, Md.).

Immunofluorescence (IF) staining. After four weeks of incubation, the cell-laden hydrogels were fixed in 4% paraformaldehyde for 30 min at room temperature, dehydrated in serial concentrations of Ethanol, paraffin-embedded and sectioned. For IF-staining, the sectioned slides were deparaffinized with Xylene, rehydrated in serial dilutions of Ethanol, and washed three times in PBST (PBS containing 0.1% Tween 20). Blocking buffer composed of 10% Goat serum, 1% BSA, 0.3% Triton X-100 in PBS was applied to the slides for 1 hour at room temperature to block unspecific binding of the antibodies. After completion of the blocking step, the slides were washed three times in PBST and incubated with the primary antibody in a humidified chamber overnight at 4° C. Then, the slides were washed three times with PBST and incubated with Alexa Fluor® 488 secondary antibody in the dark for 1 hour at room temperature. In the final step, the slides were counter-stained with DAPI (1 μg/ml). The primary and secondary antibodies are listed in Table 2. The percentage of positive cells was calculated using Image J software.

TABLE 2
Primary Antibodies for immunofluorescence staining

| Antibody | Supplier | Dilution |
|---|---|---|
| SOX2 | Proteintech | 1:200 |
| GATA3 | Proteintech | 1:200 |
| PAX2 | Invitrogen | 1:100 |
| PAX8 | Proteintech | 1:500 |
| Myo7a | Proteintech | 1:100 |

Subcutaneous implantation of encapsulated GMSCs. All of the in vivo surgical procedures were performed in accordance with established animal protocols and according to methods described previously [S. Ansari, I. M. Diniz, C. Chen, P. Sarrion, A. Tamayol, B. M. Wu, A. Moshaverinia, Human Periodontal Ligament- and Gingiva-derived Mesenchymal Stem Cells Promote Nerve Regeneration When Encapsulated in Alginate/Hyaluronic Acid 3D Scaffold, Adv. Healthc. Mater. 6 (2017) 1700670. doi:10.1002/adhm.201700670]. The alginate hydrogel containing 20% Matrigel (Alg-20MG) was selected as the optimum composition for the auditory progenitor induction based on the in vitro studies. Briefly, 4×10⁶ GMSCs were encapsulated in Alg-20MG microspheres loaded with the necessary growth factors (bFGF, IGF-I, and EGF). Cell-free hydrogels served as the negative control. Approximately 10 spheres were implanted subcutaneously under the dorsal surface of 5-month-old nude mice (n=7). One month after implantation, the mice were sacrificed, and the retrieved microspheres were utilized for studying the expression of the auditory progenitor cell markers by immunofluorescence staining analysis.

Statistical analysis. Quantitative data are expressed as mean±standard deviation (SD) from at least three independent experiments. Differences between experimental and control groups were analyzed by a two-tailed Student's t test or one-way ANOVA as appropriate. Values of p<0.05 were considered statistically significant.

### Example 1. Isolation and Characterization of Gingival Mesenchymal Stem Cells

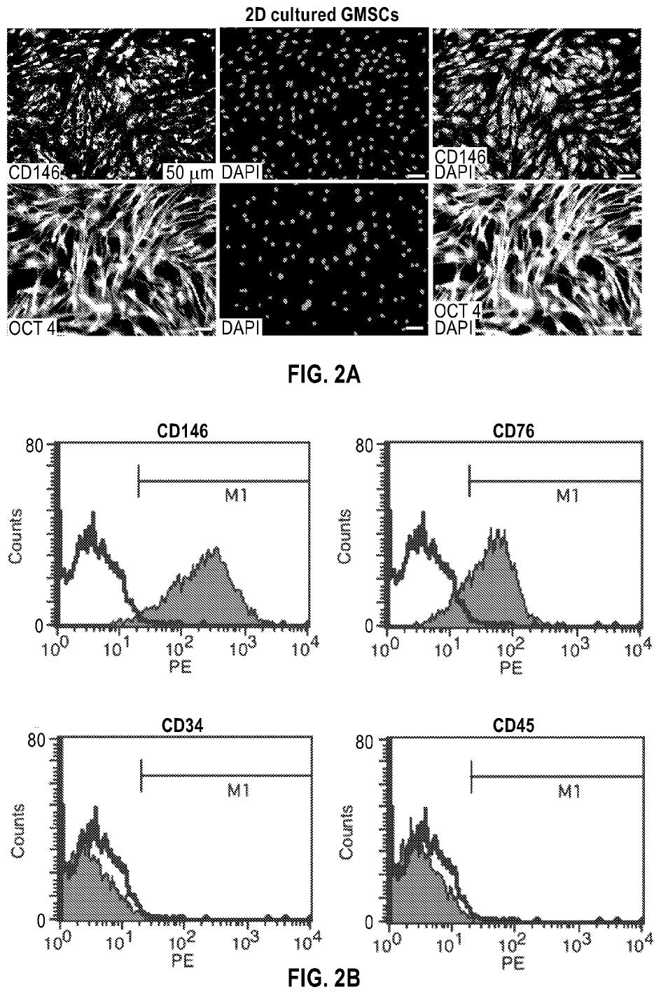

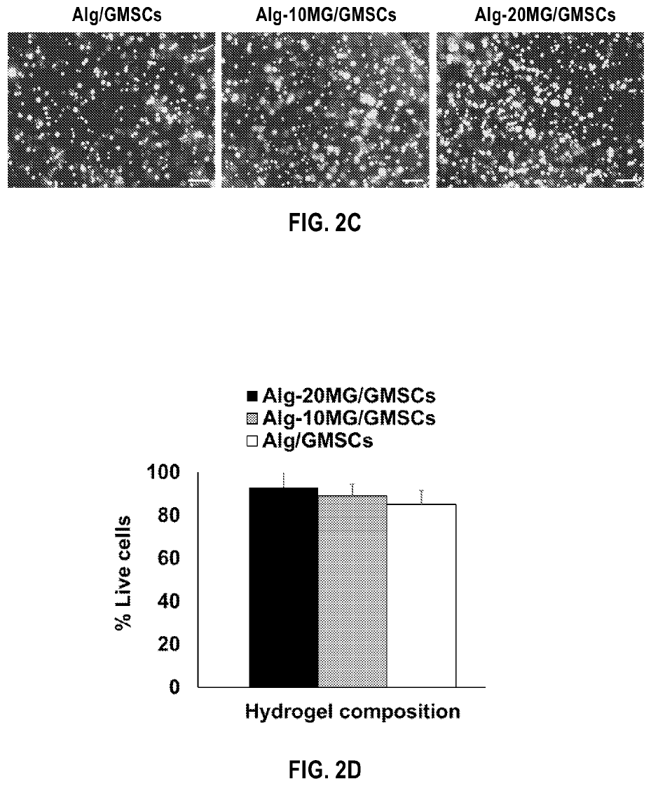

In this study, we assessed the potential of GMSCs to give rise to auditory progenitor cells and evaluated the effect of the encapsulating material on auditory progenitor induction. FIG. 1 depicts a schematic illustration of isolating GMSCs from gingival tissue, expanding the cells, and encapsulating them within the synthesized alginate-Matrigel hydrogel for auditory progenitor regeneration. Immunofluorescent (IF) staining revealed expression of the stemness markers OCT4 and CD146 (FIG. 2A). Stemness of the isolated cells was further confirmed by analyzing the expression of MSC-specific surface markers like CD-146 by flow cytometric analysis (FIG. 2B).

The results of the live/dead assay indicated that the cells were highly viable (>85%) in all experimental groups after a week of encapsulation (FIGS. 2C and 2D). No significant difference was observed among the different groups (p>0.5).

### Example 2. Characterization of the Fabricated Hydrogel Scaffold

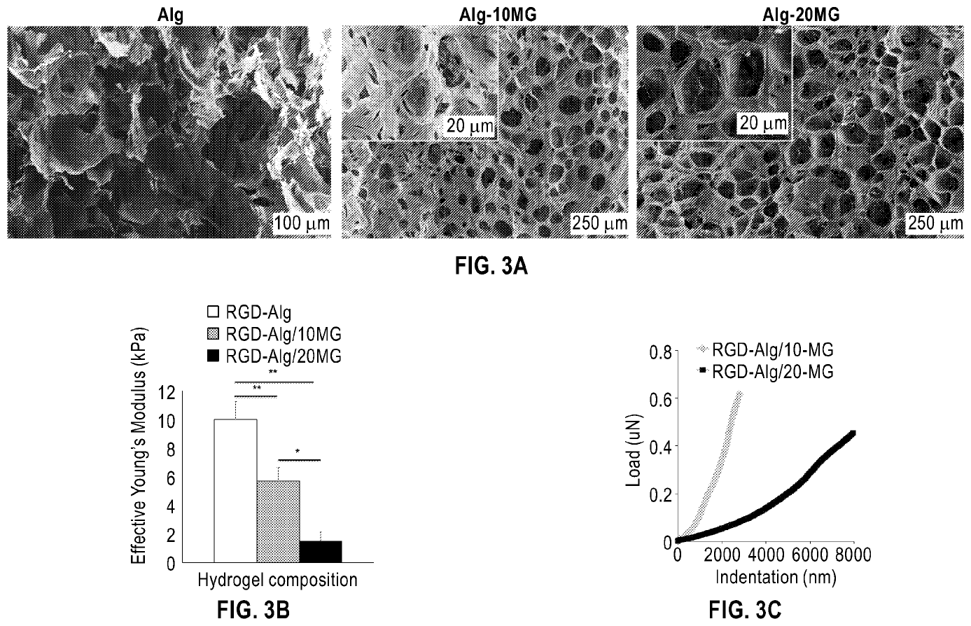

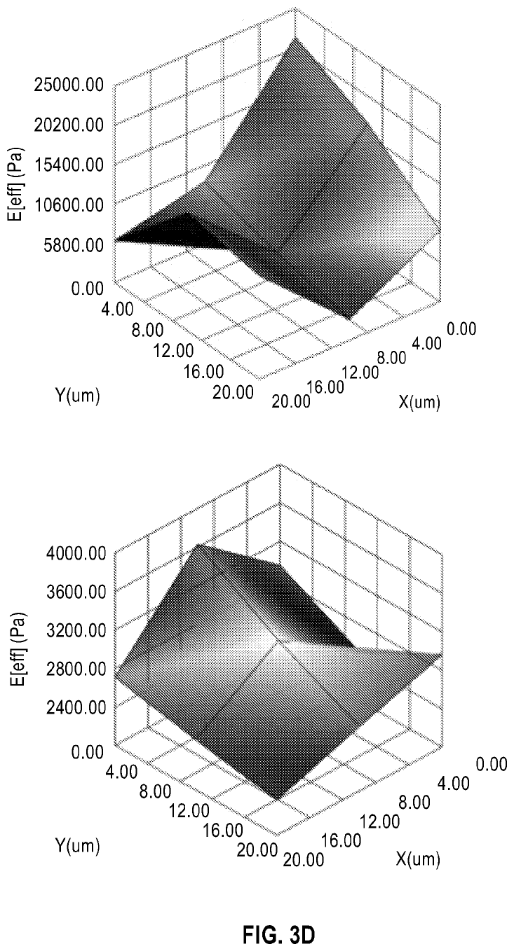

The results of SEM imaging showed that fibers of Matrigel made a homogeneous interpenetrating network inside the RGD-Alginate hydrogel (FIG. 3A). The average pore size of the fabricated hydrogels was about 100 μm. The calculated effective Young's Moduli of the Alg-20MG and Alg-10MG hydrogels were 1.52 kPa and 5.7 kPa, respectively (FIG. 3B). The Young's modulus of the cross-linked RGD-Alginate without Matrigel was about 10 kPa. The Force vs. Displacement graph obtained from indenting the cross-linked hydrogels is shown in FIG. 3C. Additionally, FIG. 3D shows elasticity map of the Alginate-Matrigel nanocomposite hydrogels.

### Example 3. In Vitro Differentiation Potential of GMSCs

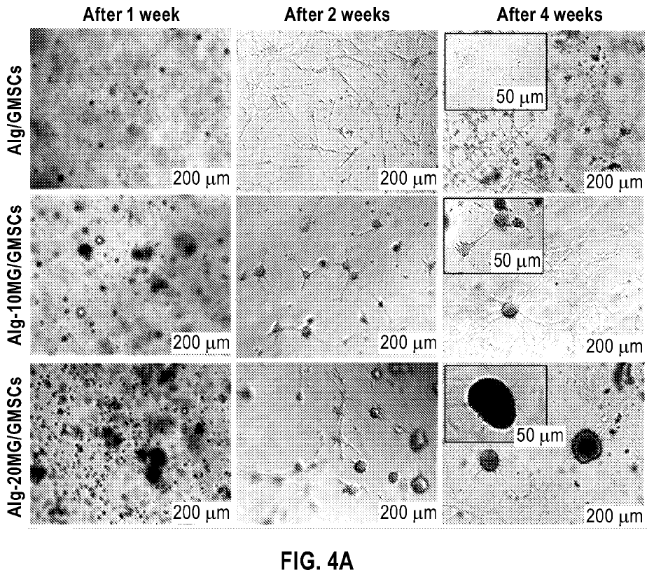

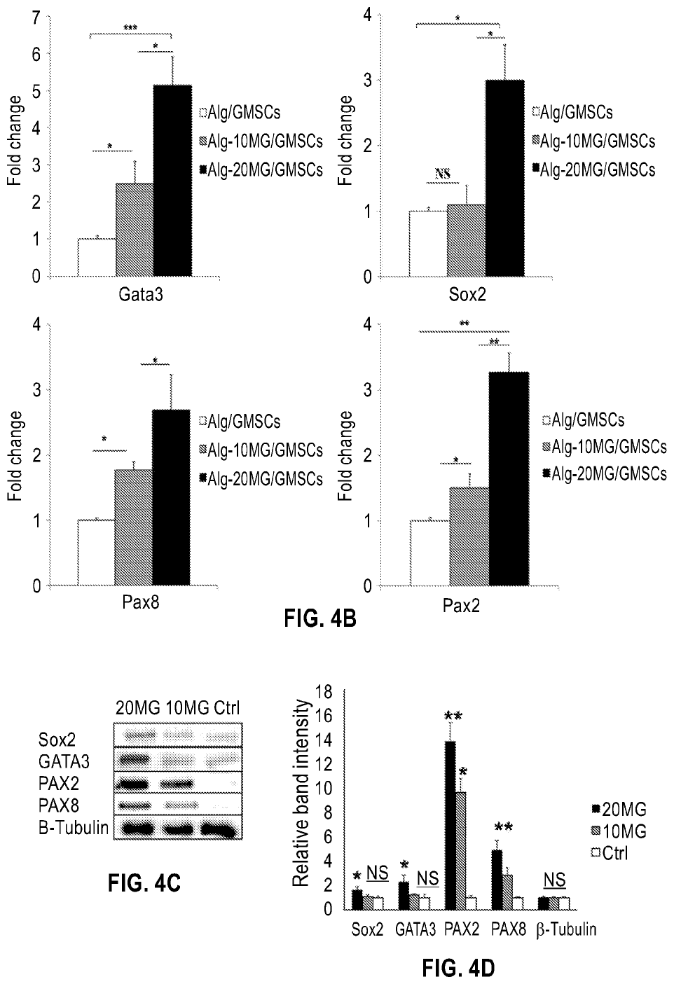

The encapsulated cells started forming small neurospheres after a week of incubation in the auditory progenitor induction medium (FIG. 4A). The size and number of neurospheres increased over time. As shown in FIG. 4A, cells encapsulated in Alg-20MG (the formulation with lower elasticity) had bigger and more numerous neurospheres in comparison to the cells encapsulated in Alg-10MG. We could rarely find neurospheres in the hydrogels formulated without Matrigel. After four weeks of culture, we could see that the GMSCs encapsulated in both Alg-10MG and Alg-20MG had neuron-like morphologies with long protrusions. Moreover, we found more mature neurospheres in the cell-laden hydrogels with the higher concentration of Matrigel (Alg-20MG), as identified by longer and more numerous protrusions.

### Example 4. Gene and Protein Expression Studies

Results of the gene expression studies after 4 weeks of incubation in the auditory progenitor inductive culture media suggested that, although cell encapsulated in both alginate-based hydrogels showing the same trends, the preplacodal ectoderm markers GATA3 and SOX2 had stronger expression in the GMSCs encapsulated in Alg-20MG in comparison to the cells encapsulated in Alg-10MG or in alginate without Matrigel (control) (FIG. 4B). Along with these results, our experiments showed higher expression of early otic markers PAX2 and PAX8 in Alg-20MG in comparison to Alg-10MG. None of these genes were highly expressed in the control group. Moreover, as shown in FIG. 4C, Western blot studies confirmed the expression of auditory progenitor markers at the protein level. The relative intensity of the acquired bands was quantified relative to those of the control group (Alginate hydrogel without Matrigel). GATA3, SOX2, PAX2 and PAX8 bands were observed in both experimental groups; however, the intensities of the bands were significantly higher in the Alg-20MG group than in the Alg-10MG or control groups, confirming the higher auditory progenitor regenerative potential of the GMSCs in presence of a higher concentration of Matrigel (FIG. 4D). Data are representative of at least three separate experiments.

### Example 5. Immunofluorescence (IF) Staining

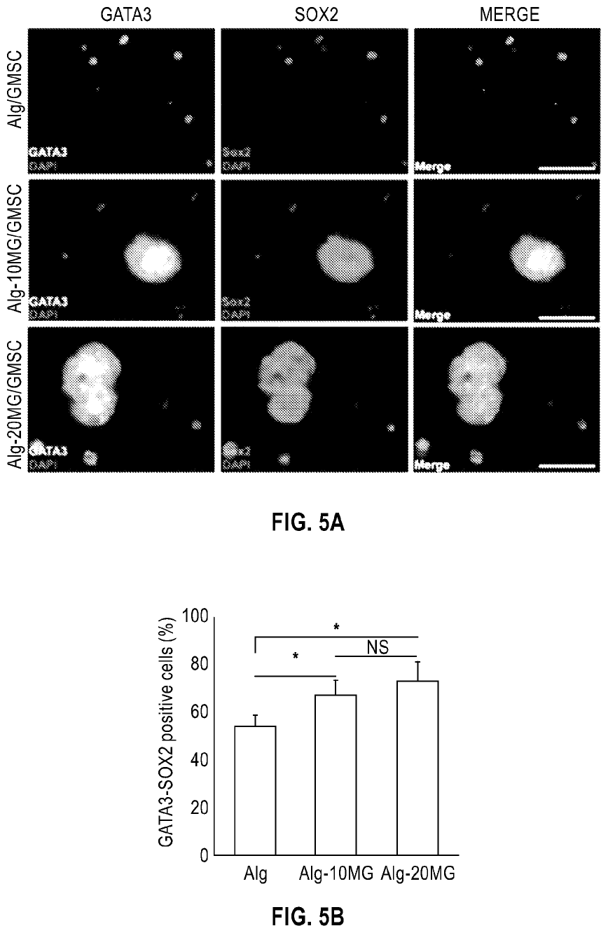

The results of IF staining demonstrated that the preplacodal ectoderm markers GATA3 and SOX2 were expressed in all of the experimental groups but the GMSCs encapsulated in Alg-20MG had higher expression levels in comparison to the cells encapsulated in Alg-10MG or without Matrigel (control) (FIG. 5A). Semi-quantitative analysis of the GATA3-SOX2 double positive staining demonstrated significantly higher level of expression in the Alg-20MG samples compared to the Matrigel-free samples but not significant differences compared to the Alg-10MG group (FIG. 5B).

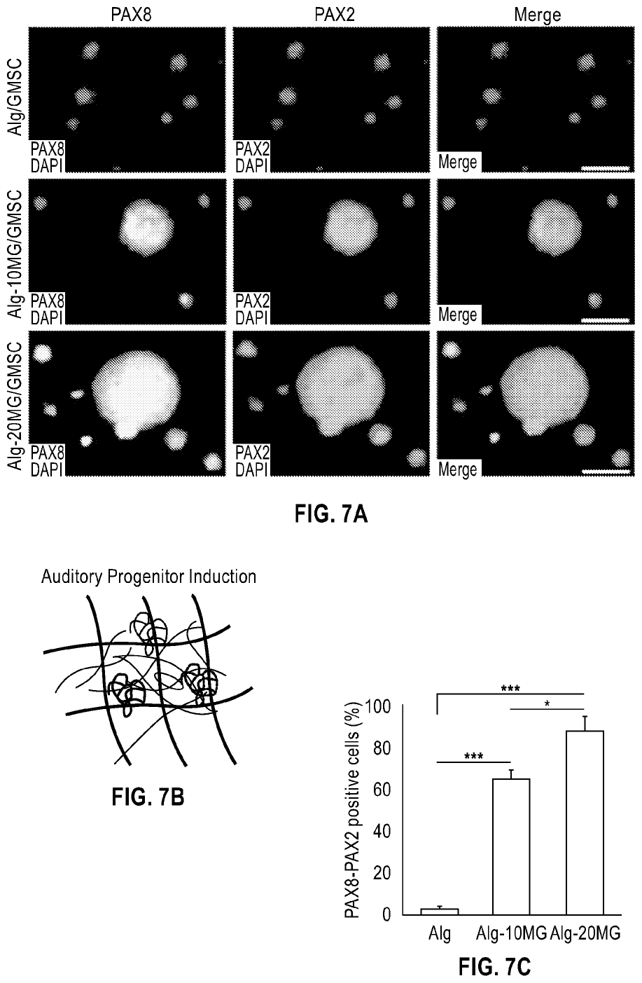

Moreover, early otic markers PAX2 and PAX8 could not be detected in the absence of Matrigel. However, these markers were expressed at higher levels in the Alg-20MG and a lower level in the Alg-10MG samples (FIG. 7A). These results confirmed the gene and protein expression observations. The schematic illustration in FIG. 7B shows encapsulation of the GMSCs in alginate-Matrigel nanohybrid hydrogel in presence of necessary growth factors (FIG. 7B). Semi-quantitative analysis of the PAX8-PAX2 double positive staining demonstrated significantly higher level of expression in the Alg-20MG samples compared to the Alg-10MG and the Matrigel-free samples (FIG. 7C).

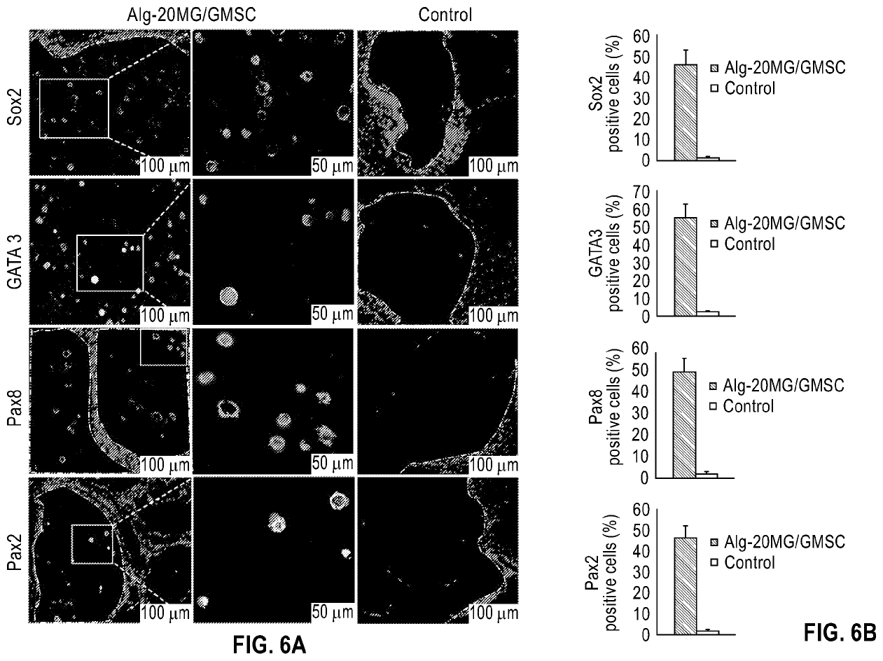

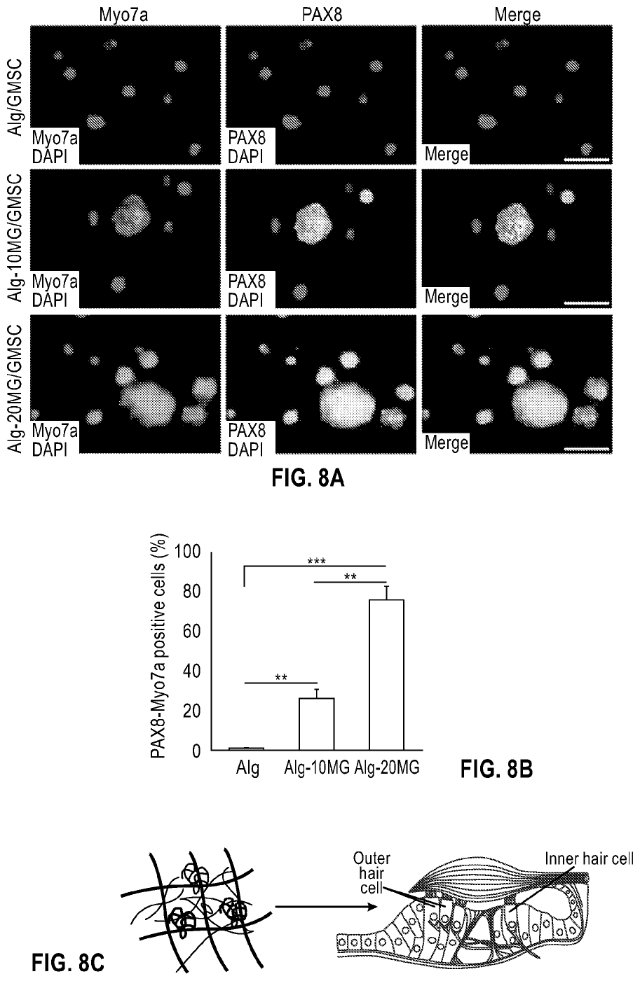

Interestingly, Myo7a, a sensory auditory cell marker, was highly expressed in the cells encapsulated in Alg-20MG but had lower expression in the cells encapsulated in the Alg-10MG (FIG. 8A). Like other markers, this marker was also not expressed in the absence of Matrigel. As FIG. 6a and the semi-quantitative analysis in FIG. 8B demonstrate, Myo7a⁺Pax8⁺ double-positive staining indicated successful otic induction in the GMSCs encapsulated in the Alg-20MG, in vitro. The image in FIG. 8C illustrates the successful differentiation of the encapsulated GMSCs schematically.

### Example 6. Encapsulated GMSCs Contribute to Auditory Progenitors In Vivo

To study the inductive potential of the alginate-Matrigel composite hydrogel and the growth factors EGF, IGF, and bFGF on the differentiation of the encapsulated GMSCs in vivo, we utilized a subcutaneous implantation model in immunocompromised mice. Immunofluorescent staining was used to evaluate the ability of the hydrogel to produce auditory progenitor induction of GMSCs in vivo. As FIG. 6A shows, the GMSCs could successfully differentiate into progenitors expressing GATA3, SOX2, PAX2, and PAX8in vivo in the presence of the necessary signaling factors. The semi-quantitative analysis demonstrated over 45% positively stained cells encapsulated in Alg-20MG hydrogel (FIG. 6B). The cell-free hydrogels did not demonstrate any positive staining for the tested markers.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating hearing loss associated with loss of sensory neurons in a human subject, the method comprising:
a. obtaining a population of gingival mesenchymal stem cells (GMSCs);
b. optionally expanding the population of GMSCs in vitro;
c. encapsulating the population of GMSCs in an elastic three-dimensional scaffold;
d. exposing the encapsulated population of GMSCs to a composition comprising one or more growth factors;
e. allowing a sufficient period for the population of GMSCs to differentiate towards auditory progenitor cells;
f. optionally retrieving the auditory progenitor cells from the scaffold; and
g. introducing the auditory progenitor cells into the inner ear of the subject.

2. The method of claim 1 wherein the population of GMSCs are obtained from gingival tissue of a donor.

3. The method of claim 2 wherein the donor is the human subject.

4. The method of claim 1 wherein the elasticity of the scaffold is a Young's modulus between about 1 kPa and about 30 kPa.

5. The method of claim 4 wherein the elasticity of the scaffold is a Young's modulus is between about 1 kPa and about 12 kPa.

6. The method of claim 4 wherein the elasticity of the scaffold is a Young's modulus is between about 1 kPa and about 6 kPa.

7. The method of claim 1 wherein the elasticity of the scaffold is equal to or below an elastic modulus calculated from the slope of the initial portion of the loading curve of RGD-ALG/10-MG depicted in FIG. 3C.

8. The method of claim 1 wherein the elasticity of the scaffold is equal to or below an elastic modulus calculated from the slope of the initial portion of the loading curve of RGD-ALG/20-MG depicted in FIG. 3C.

9. The method of claim 1 wherein the one or more growth factors are selected from basic fibroblast growth factor (bFGF), insulin-like growth factor 1 (IGF-1), epidermal growth factor (EGF), BMP4, FGF-2, SB-431542, CHIR99021, LDN-193189 or any combination thereof.

10. The method of claim 1 wherein the one or more growth factors are a combination of basic fibroblast growth factor (bFGF), insulin-like growth factor 1 (IGF-1), and epidermal growth factor (EGF).

11. The method of claim 1 wherein the scaffold comprises a hydrogel.

12. The method of claim 11 wherein the hydrogel is a nanocomposite hydrogel.

13. The method of claim 11 wherein the hydrogel comprises RGD peptide.

14. The method of claim 11 wherein the hydrogel comprises alginate crosslinked with a divalent cation.

15. The method of claim 11 wherein the hydrogel comprises RGD-alginate.

16. The method of claim 11 wherein the hydrogel comprises MATRIGEL.

17. The method of claim 16 wherein the hydrogel comprises about at least 10% MATRIGEL.

18. The method of claim 17 wherein the hydrogel comprises about 10% to about 20% MATRIGEL.

19. The method of claim 11 wherein the hydrogel comprises RDG-alginate and at least 10% MATRIGEL.

20. The method of claim 19 wherein the hydrogel comprises RDG-alginate and about 10% to about 20% MATRIGEL.

21. The method of claim 1 wherein the GMSCs lack expression of CD34 or CD45, express CD76, CD146, or OCT4, or any combination thereof.

22. The method of claim 1 wherein the auditory progenitor cells express GATA3, SOX2, PAX2, PAX8, or any combination thereof.

23. The method of claim 1 wherein the auditory progenitor cells are injected into the auditory nerve trunk in the internal auditory meatus of the human subject in need thereof, thereby treating the hearing loss in the subject.

24. An in vitro method of generating auditory progenitor cells, the method comprising the steps of:
a. obtaining a population of gingival mesenchymal stem cells (GMSCs);
b. optionally expanding the population of GMSCs in vitro;
c. encapsulating the population of GMSCs in an elastic three-dimensional scaffold;
d. exposing the encapsulated population of GMSCs to a composition comprising one or more growth factors;
e. allowing a sufficient period for the population of GMSCs to differentiate towards auditory progenitor cells; and
f. retrieving the auditory progenitor cells from the scaffold.

25. The method of claim 24 wherein the population of GMSCs are obtained from gingival tissue of a mammalian donor.

26. The method of claim 25 wherein the donor is human.

27. The method of claim 24 wherein the elasticity of the scaffold is a Young's modulus between about 1 kPa and about 30 kPa.

28. The method of claim 27 wherein the elasticity of the scaffold is a Young's modulus is between about 1 kPa and about 12 kPa.

29. The method of claim 27 wherein the elasticity of the scaffold is a Young's modulus is between about 1 kPa and about 6 kPa.

30. The method of claim 24 wherein the elasticity of the scaffold is equal to or below an elastic modulus calculated from the slope of the initial portion of the loading curve of RGD-ALG/10-MG depicted in FIG. 3C.

31. The method of claim 24 wherein the elasticity of the scaffold is equal to or below an elastic modulus calculated from the slope of the initial portion of the loading curve of RGD-ALG/20-MG depicted in FIG. 3C.

32. The method of claim 24 wherein the one or more growth factors are selected from basic fibroblast growth factor (bFGF), insulin-like growth factor 1 (IGF-1), epidermal growth factor (EGF), BMP4, FGF-2, SB-431542, CHIR99021, LDN-193189 or any combination thereof.

33. The method of claim 24 wherein the one or more growth factors are a combination of basic fibroblast growth factor (bFGF), insulin-like growth factor 1 (IGF-1), and epidermal growth factor (EGF).

34. The method of claim 24 wherein the scaffold comprises a hydrogel.

35. The method of claim 34 wherein the hydrogel is a nanocomposite hydrogel.

36. The method of claim 34 wherein the hydrogel comprises RGD peptide.

37. The method of claim 34 wherein the hydrogel comprises alginate crosslinked with a divalent cation.

38. The method of claim 34 wherein the hydrogel comprises RGD-alginate.

39. The method of claim 34 wherein the hydrogel comprises MATRIGEL.

40. The method of claim 34 wherein the hydrogel comprises about at least 10% MATRIGEL.

41. The method of claim 40 wherein the hydrogel comprises about 10% to about 20% MATRIGEL.

42. The method of claim 34 wherein the hydrogel comprises RDG-alginate and at least 10% MATRIGEL.

43. The method of claim 42 wherein the hydrogel comprises RDG-alginate and about 10% to about 20% MATRIGEL.

44. The method of claim 24 wherein the GMSCs lack expression of CD34 or CD45, express CD76, CD146, or OCT4, or any combination thereof.

45. The method of claim 24 wherein the auditory progenitor cells express GATA3, SOX2, PAX2, PAX8, or any combination thereof.

46. The method of claim 24 wherein the auditory progenitor cells are injected into the auditory nerve trunk in the internal auditory meatus of a human subject in need thereof, thereby treating hearing loss in the subject.

47. The method of claim 46 wherein the human subject is the donor.

48. A method for treating hearing loss in a subject comprising the steps of
a. preparing auditory progenitor cells in accordance with claim 24; and
b. introducing the auditory progenitor cells into the inner ear of the subject.

49. The method of claim 48 wherein the population of gingival mesenchymal stem cells (GMSCs) is from the subject.


SEQUENCE LISTING (10 sequences)

SEQ ID NO: 1 - DNA, 23 bp, Homo sapiens
ataataacaatcatcatcggcgg

SEQ ID NO: 2 - DNA, 20 bp, Homo sapiens
aaaaagagagaggcaaactg

SEQ ID NO: 3 - DNA, 21 bp, Homo sapiens
gctgtctgcagccaggagagc

SEQ ID NO: 4 - DNA, 22 bp, Homo sapiens
atgcatcaaacaactgtggcca

SEQ ID NO: 5 - DNA, 20 bp, Homo sapiens
aacgacagaacccgactatg

SEQ ID NO: 6 - DNA, 20 bp, Homo sapiens
atcccactgggtcattggag

SEQ ID NO: 7 - DNA, 21 bp, Homo sapiens
ctgagggcgtctgtgacaatg

SEQ ID NO: 8 - DNA, 21 bp, Homo sapiens
tgaatggttgctgcactttgg

SEQ ID NO: 9 - DNA, 21 bp, Homo sapiens
agccgcatcttcttttgcgtc

SEQ ID NO: 10 - DNA, 21 bp, Homo sapiens
tcatatttggcaggtttttct